(19) United States
(12) Patent Application Publication
(10) Pub. No.: US 2012/0148662 A1
(43) Pub. Date: Jun. 14, 2012

(54) Formulation

(76) Inventors: Guenter Gross, Weil am Rhein (DE); Joseph Tardio, Saint Louis (FR)

(21) Appl. No.: 13/311,640

(22) Filed: Dec. 6, 2011

(30) Foreign Application Priority Data
Dec. 8, 2010 (EP) 10194219.1

(51) Int. Cl. A61K 9/127; A61K 31/255

(52) U.S. Cl. 424/450; 514/513

(57) ABSTRACT

The present invention relates to a novel stable S-[2-([[1-(2-ethylbutyl)-cyclohexyl]-carbonyl]amino)phenyl]2-methylpropanethioate liposomal composition, a process for the preparation thereof and its use in the treatment of diseases.

### PRIORITY TO RELATED APPLICATION(S)

This application claims the benefit of European Patent Application No. 10194219.1, filed Dec. 8, 2010, which is hereby incorporated by reference in its entirety.

### BACKGROUND OF THE INVENTION

S-[2-([[1-(2-ethylbutyl)-cyclohexyl]-carbonyl]amino)phenyl]2-methylpropanethioate is also known as thioisobutyric acid S-(2-{[1-(2-ethyl-butyl)-cyclohexanecarbonyl]-amino}-phenyl) ester, dalcetrapib, JTT-705 or the compound of formula I:

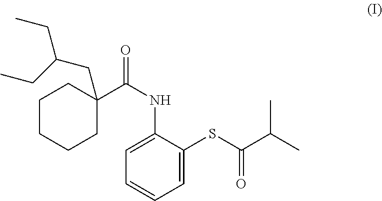

S-[2-([[1-(2-ethylbutyl)cyclohexyl]carbonyl]amino) phenyl]2-methylpropanethioate has been shown to be an inhibitor of CETP activity in humans (de Grooth et al., Circulation, 105, 2159-2165 (2002)) and rabbits (Shinkai et al., J. Alfed. Chez., 43, 3566-3572 (2000); Kobayashi et al., Atherosclerosis, 162, 131-135 (2002); and Okamoto et al., Nature, 406 (13), 203-207 (2000)). S-[2-([[1-(2-ethylbutyl)cyclohexyl]carbonyl]amino)phenyl]2-methylpropanethioate has been shown to increase plasma HDL cholesterol in humans (de Grooth et al., supra) and in rabbits (Shinkai et al., supra; Kobayashi et al., supra; Okamoto et al., supra). Moreover, S-[2-([[1-(2-ethylbutyl)cyclohexyl]carbonyl]amino)phenyl]2-methylpropanethioate has been shown to decrease LDL cholesterol in humans (de Grooth et al., supra) and rabbits (Okamoto et al., supra). Additionally, S-[2-([[1-(2-ethylbutyl)cyclohexyl]carbonyl]amino)phenyl]2-methylpropanethioate inhibits the progression of atherosclerosis in rabbits (Okamoto et al., supra). S-[2-([[1-(2-ethylbutyl)cyclohexyl]carbonyl]amino)phenyl]2-methylpropanethioate, as well as methods of making and using the compound, are described in EP patent EP1020439, Shinkai et al., J. Med. Chem. 43:3566-3572 (2000), WO 2007/051714, and WO 2008/074677. However, International Patent Application WO2004082593 recognizes that S-[2-([[1-(2-ethylbutyl)-cyclohexyl]-carbonyl]amino)phenyl]2-methylpropanethioate has extremely low aqueous solubility, and has low oral bioavailability when dosed conventionally. Thus, a need exists for improved formulations which increase the aqueous solubility and oral bioavailability of this compound and other similar compounds.

### SUMMARY OF THE INVENTION

The present invention relates to a novel stable S-[2-([[1-(2-ethylbutyl)-cyclohexyl]-carbonyl]amino)phenyl]2-methylpropanethioate liposomal composition, a process for the preparation thereof and its use in the treatment of diseases.

### DETAILED DESCRIPTION OF THE INVENTION

Unless otherwise stated, the following terms used in the specification and claims have the meanings given below:

The term “liposome” as used herein relates to a spherical vesicle with a membrane comprising a phospholipid bilayer. The term lipid as used herein relate to an amphiphilic class of hydrocarbon-containing organic compounds.

The term “pharmaceutically acceptable water miscible solvent” or “pharmaceutically acceptable solvent” includes ethanol, glycolfurol, propyleneglycol, or a mixture thereof, in particular ethanol.

The term “vesicle”, as used herein, relates to a small and enclosed compartment, which comprises at least one membrane enclosing the compartment. The term “compartment” relates to the core of the vesicle. The membrane separates the content of the core from the outside environment of the vesicle. The term membrane as used herein refers to a lipid bilayer enclosing a compartment.

The present invention provides a pharmaceutical composition that results in increased bioavailability of S-[2-([[1-(2-ethylbutyl)-cyclohexyl]-carbonyl]amino)phenyl]2-methylpropanethioate, and its use in the treatment of diseases, particularly cardiovascular disorders. The drug is administered in a dissolved form which avoids any barriers due to solubility or dissolution limited absorption.

In particular, the invention is directed to a stable composition comprising S-[2-([[1-(2-ethylbutyl)-cyclohexyl]-carbonyl]amino)phenyl]2-methylpropanethioate and liposomes, wherein the S-[2-([[1-(2-ethylbutyl)-cyclohexyl]-carbonyl]amino)phenyl]2-methylpropanethioate is substantially entrapped in a liposome membrane. Specifically, at least about 95% of S-[2-([[1-(2-ethylbutyl)-cyclohexyl]-carbonyl]amino)phenyl]2-methylpropanethioate in the composition is entrapped in the liposome. In particular embodiments, 100% of S-[2-([[1-(2-ethylbutyl)-cyclohexyl]-carbonyl]amino)phenyl]2-methylpropanethioate in the composition is entrapped in the liposome.

In a particular embodiment, the present invention comprises a composition as described above wherein the liposomes have sizes of about 20 to about 1000 nm, in particular about 25 to about 200 nm. In more particular embodiments, the present invention comprises a composition as described above wherein about 80%, and more particularly about 95%, of the liposomes have sizes of about 25 nm to about 200 nm.

In other particular embodiments, the present invention comprises a composition as described above wherein the liposomes are 95% egg lecithin or soybean lecithin.

In other particular embodiments, the present invention provides a composition wherein lecithin and at least one stabilizer form the liposome.

In a particular embodiment, the present invention further provides a composition comprising at least 40% of water.

In a further embodiment, the present invention provides a composition wherein 0.01% to 0.5%, particularly 0.1% to 0.3%, and more particularly 0.25% by weight per volume, of S-[2-([[1-(2-ethylbutyl)-cyclohexyl]-carbonyl]amino)phenyl]2-methylpropanethioate is present.

The liposome components of these pharmaceutical compositions are lipids and, in particular, phospholipids. In particular, the phospholipids are the lecithins. The lecithin can be of vegetable, animal or synthetic origin; as for example, soybean lecithin, egg lecithin or L-β-oleoyl-2-palmitoyl-α-lecithin, and more particularly egg lecithin.

In certain embodiments of the present invention as defined herein the composition is in the form of a solution. In more specific embodiments of the present invention as defined herein the composition is in the form of an aqueous solution.

In a particular embodiment, the present invention is parenterally administrated.

In a further embodiment, the present invention provides a composition for treating or preventing cardiovascular disorder.

In another embodiment, the invention provides a kit comprising:

a solution of S-[2-([[1-(2-ethylbutyl)-cyclohexyl]-carbonyl]amino)phenyl]2-methylpropanethioate in alcohol; and

a solution of liposome, in particular lecithin, optionally with at least one stabilizer.

In another embodiment, the invention provides a kit comprising:

a solution of S-[2-([[1-(2-ethylbutyl)-cyclohexyl]-carbonyl]amino)phenyl]2-methylpropanethioate in alcohol, particularly wherein the alcohol is ethanol, glycolfurol, propyleneglycol, or a mixture thereof, more particularly ethanol; and

a solution of liposome, particularly wherein the liposome is a spherical vesicle with a membrane comprising a phospholipid bilayer, more particularly wherein the phospholipid is lecithin, and optionally with at least one stabilizer.

In another embodiment the invention provides a kit comprising:

a solution of S-[2-([[1-(2-ethylbutyl)-cyclohexyl]-carbonyl]amino)phenyl]2-methylpropanethioate in alcohol, particularly wherein the alcohol is ethanol, glycolfurol, propyleneglycol, or a mixture thereof, more particularly ethanol; and

an aqueous solution of liposome, particularly wherein the liposome is a spherical vesicle with a membrane comprising a phospholipid bilayer, more particularly wherein the phospholipids is lecithin, and optionally with at least one stabilizer.

In further embodiments, the invention provides a kit comprising:

a vial with a solution of S-[2-([[1-(2-ethylbutyl)-cyclohexyl]-carbonyl]amino)phenyl]2-methylpropanethioate in a pharmaceutically acceptable solvent; and

a vial with a solution of liposome, in particular lecithin, optionally with at least one stabilizer.

In further embodiments, the invention provides a kit comprising:

a vial with a solution of S-[2-([[1-(2-ethylbutyl)-cyclohexyl]-carbonyl]amino)phenyl]2-methylpropanethioate in a pharmaceutically acceptable solvent; particularly wherein the pharmaceutically acceptable solvent is ethanol, glycolfurol, propyleneglycol, or a mixture thereof, more particularly ethanol; and

a vial with a solution of liposome, particularly wherein the liposome is a spherical vesicle with a membrane comprising a phospholipid bilayer, more particularly wherein the phospholipids is lecithin, and optionally with at least one stabilizer.

In further embodiments, the invention provides a kit comprising:

a vial with a solution of S-[2-([[1-(2-ethylbutyl)-cyclohexyl]-carbonyl]amino)phenyl]2-methylpropanethioate in pharmaceutically acceptable solvent; particularly wherein the pharmaceutically acceptable solvent is ethanol, glycolfurol, propyleneglycol, or a mixture thereof, more particularly ethanol; and

a vial with a solution of liposome in water, particularly wherein the liposome is a spherical vesicle with a membrane comprising a phospholipid bilayer, more particularly wherein the phospholipids is lecithin, and optionally with at least one stabilizer.

In another embodiment, the present invention provides a method of preparing a liposome composition comprising:

a) preparing an oil-soluble composition comprising S-[2-([[1-(2-ethylbutyl)-cyclohexyl]-carbonyl]amino)phenyl]2-methylpropanethioate and lecithin in a pharmaceutically acceptable solvent;
b) preparing a water soluble composition comprising a stabilizer and water;
c) combining the water soluble composition with the oil-soluble composition obtained according to step b) and a) respectively;
d) stirring in particular at 200 to 500 rpm e.g. with a magnetic stirrer; and
e) homogenizing at high pressure, particularly between 200 to 1500 atm (50 to 80 MPa), more particularly between 500 to 800 atm (50 to 80 MPa).

In another embodiment, the present invention provides a method of preparing a liposome composition comprising:

a) preparing an oil-soluble composition comprising S-[2-([[1-(2-ethylbutyl)-cyclohexyl]-carbonyl]amino)phenyl]2-methylpropanethioate and lecithin in a pharmaceutically acceptable solvent;
b) preparing a water soluble composition comprising Mannitol and water;
c) combining the water soluble composition with the oil-soluble composition obtained according to step b) and a) respectively;
d) stirring in particular at 500 rpm e.g. with a magnetic stirrer;
e) homogenizing at a pressure between 500 to 800 atm (50 to 80 MPa); and
f) filtering the liposome solution.

In another embodiment, the present invention provides a method of preparing a liposome composition comprising:

a) preparing an oil-soluble composition comprising S-[2-([[1-(2-ethylbutyl)-cyclohexyl]-carbonyl]amino)phenyl]2-methylpropanethioate and lecithin in 1% to 10% of pharmaceutically acceptable solvent;
b) preparing a water soluble composition comprising mannitol and water;
c) combining the water soluble composition with the oil-soluble composition obtained according to step b) and a) respectively;
d) stirring in particular at 500 rpm, e.g. with a magnetic stirrer; and
e) homogenizing at high pressure, particularly between 200 to 1500 atm (20 MPa to 150 MPa), and more particularly between 500 to 800 atm (50 MPa to 80 MPa).

In another embodiment, the present invention can be sterilized, spray-dried and/or lyophilised. In a particular embodiment, the present invention can be steam sterilized.

The vesicles hereinbefore described may comprise multiple layers, each of which comprises the ingredients listed above. Theses liposomes are also known as oligolamellar or multilamellar vesicles. Anderson et al. described these vesicles in which proteins drugs are encapsulated (1994, CYTOKINE 6, p92-101). The term multi-lamellar liposome as used herein relates to a liposome with a multiple layer structure wherein said layers are separated by aqueous medium. The vesicles may comprise a phospholipid bilayer.

Said phospholipids may be selected from one or more phospholipids of the group consisting of egg phosphatidylethanolamine, egg lecithin, dipalmitoyl lecithin, lecithin, egg phosphatidylcholine, dioleoyl phosphatidylcholine, 1-palmitoyl-2-oleoyl-sn-glycerol-3-phosphatidylcholine, dipalmitoylphosphatidylcholine, dimyristoylphosphatidylcholine and long-chain or intermediate-chain phosphatidylcholine, in particular egg or soybean lecithin. More particularly the phospholipid is egg lecithin.

Another ingredient of the pharmaceutical composition of this invention is a stabilizer, in particular a carbohydrate, which functions as a protective colloid and provides long-term stability to the compositions. The sugar component can be the usual monosaccharides and disaccharides or it can be a sugar-like polyol. Examples of suitable sugar components include glucose, fructose, sucrose, sorbitol, mannitol and xylitol. In particular, the sugar is sucrose or sorbitol. In particular embodiments, the sugar is mannitol. Stabilizers include 1% to 25% carbohydrates (e.g. mannitol, sorbitol, sucrose, cellulose derivative) and/or 0.5% to 3% charged phospholipids (e.g. phosphatidylglycerol, phosphatidic acid, phosphatidylserine) and/or cholesterol. In particular embodiments, stabilizers include 1% to 25% by weight per volume of carbohydrates (e.g. mannitol, sorbitol, sucrose, cellulose derivative) and/or 0.5% to 3% by weight per volume of charged phospholipids (e.g. phosphatidylglycerol, phosphatidic acid, phosphatidylserine) and/or cholesterol.

The liposome compositions of this invention can also contain pharmaceutical adjuvants. Examples of such optional pharmaceutical adjuvants include those substances which are usual in compositions such as small amounts of other lipids, e.g. cholesterol, antioxidants, synergists, preserving agents, stabilizing agents, buffers for adjusting to the desired pH value or agents for adjusting the osmotic pressure. The required and optimum amounts of these pharmaceutical adjuvants can vary with the specific compositions.

The vesicles may additionally also comprise a neutral lipid. Said neutral lipid may be a monoglyceride. Such a monoglyceride may be a middle-chain monoglyceride.

Furthermore, all embodiments of the vesicles described above may alternatively be comprised of pegylated lecithins.

The concentration of the liposome component in the solution generally lies in the range of form about 1% to about 25% (weight/volume) and preferably between about 5% and about 15% weight/volume.

When the liposome comprises multiple layers, the outermost layer will become unstable first, optionally followed by the next layer depending on carbohydrate concentration. The depot function of multi-layer liposomes is well known (Katre et al., Am J Drug Deliv 2004, 2 (4), p 213-227).

The pharmaceutical compositions of the present invention can be used to treat or prevent a cardiovascular disorder, including, but not limited to, atherosclerosis, peripheral vascular disease, dyslipidemia (e.g., hyperlipidimia), hyperbetalipoproteinemia, hypoalphalipoproteinemia, hypercholesterolemia, hypertriglyceridemia, familial-hypercholesterolemia, angina, ischemia, cardiac ischemia, stroke, myocardial infarction, reperfusion injury, angioplastic restenosis, hypertension, cardiovascular disease, coronary heart disease, coronary artery disease, hyperlipidoproteinemia, vascular complications of diabetes, obesity or endotoxemia in a mammal, especially a human (i.e., a male or female human).

Accordingly, the invention provides a method for the treatment or prophylaxis of a cardiovascular disorder in a mammal, which method comprises administering to a mammal (preferably a mammal in need thereof) a therapeutically effective amount of the pharmaceutical composition. The mammal preferably is a human (i.e. a male or female human). The human can be of any race (e.g., Caucasian or Oriental). The cardiovascular disorder preferably is selected from the group consisting of atherosclerosis, peripheral vascular disease, dyslipidemia, hyperbetalipoproteinemia, hypoalphalipoproteinemia, hypercholesterolemia, hypertriglyceridemia, familial-hypercholesterolemia, angina, ischemia, cardiac ischemia, stroke, myocardial infarction, reperfusion injury, angioplastic restenosis, hypertension, vascular complications of diabetes, obesity, and endotoxemia in a mammal. More preferably, the cardiovascular disorder is selected from the group consisting of cardiovascular disease, coronary heart disease, coronary artery disease, hypoalphalipoproteinemia, hyperbetalipoproteinemia, hypercholesterolemia, hyperlipidemia, atherosclerosis, hypertension, hypertriglyceridemia, hyperlipidoproteinemia, peripheral vascular disease, angina, ischemia, and myocardial infarction.

In certain embodiments of the present invention, the composition is a pharmaceutical composition.

The pharmaceutical composition can be, for example, in the form of a pill, capsule or tablet, each containing a predetermined amount of S-[2-([[1-(2-ethylbutyl)-cyclohexyl]-carbonyl]amino)phenyl]2-methylpropanethioate and in particular coated for ease of swallowing, in the form of a powder or granules. In particular, the pharmaceutical composition is in the form of a tablet comprising S-[2-([[1-(2-ethylbutyl)-cyclohexyl]-carbonyl]amino)phenyl]2-methylpropanethioate and the components of the tablet utilized and described therein. For oral administration, fine powders or granules may contain diluting, dispersing and/or surface active agents and may be present, for example, in capsules or sachets in the dry state, or in tablets wherein binders and lubricants may be included. Components such as sweeteners, flavoring agents, preservatives, suspending agents, thickening agents, and/or emulsifying agents also may be present in the pharmaceutical composition.

In certain embodiments of the present invention, the composition comprises 100 mg to 600 mg of S-[2-([[1-(2-ethylbutyl)-cyclohexyl]-carbonyl]amino)phenyl]2-methylpropanethioate. In particular, the composition comprises 150 mg to 450 mg of S-[2-([[1-(2-ethylbutyl)-cyclohexyl]-carbonyl]amino)phenyl]2-methylpropanethioate. More particularly, the composition comprises 250 mg to 350 mg of S-[2-([[1-(2-ethylbutyl)-cyclohexyl]-carbonyl]amino)phenyl]2-methylpropanethioate. Most particularly, the composition comprises 250 mg to 350 mg of S-[2-([[1-(2-ethylbutyl)-cyclohexyl]-carbonyl]amino)phenyl]2-methylpropanethioate.

In another embodiment of the present invention, the composition comprises for pediatric use 25 mg to 300 mg of S-[2-([[1-(2-ethylbutyl)-cyclohexyl]-carbonyl]amino)phenyl]2-methylpropanethioate. In particular the pediatric composition comprises 75 mg to 150 mg of S-[2-([[1-(2-ethylbutyl)-cyclohexyl]-carbonyl]amino)phenyl]2-methylpropanethioate.

S-[2-([[1-(2-ethylbutyl)-cyclohexyl]-carbonyl]amino)phenyl]2-methylpropanethioate can be administered to the mammal at any suitable dosage (e.g., to achieve a therapeutically effective amount). For example, a suitable dose of a therapeutically effective amount of Compound I for administration to a patient will be between approximately 100 mg to about 1800 mg per day. A desirable dose is preferably about 300 mg to about 900 mg per day. A preferred dose is about 600 mg per day.

In another embodiment, the invention provides a kit comprising a pharmaceutical composition comprising a therapeutically effective amount of S-[2-([[1-(2-ethylbutyl)-cyclohexyl]-carbonyl]amino)phenyl]2-methylpropanethioate and liposomes, prescribing information also known as a “leaflet”, a blister package or bottle (HDPE or glass) and a container. The prescribing information preferably includes the advice to a patient regarding the administration of the S-[2-([[1-(2-ethylbutyl)-cyclohexyl]-carbonyl]amino)phenyl]2-methylpropanethioate with food, especially to improve the bioavailability of S-[2-([[1-(2-ethylbutyl)-cyclohexyl]-carbonyl]amino)phenyl]2-methylpropanethioate.

Unless otherwise stated all percentages are given in weight percent of the total weight of the composition.

The following examples illustrate methods of preparation and properties of the liposomal S-[2-([[1-(2-ethylbutyl)-cyclohexyl]-carbonyl]amino)phenyl]2-methylpropanethioate composition according to the invention. Other features and embodiments of the invention will become apparent from the following examples which are given for illustration of the invention rather than for limiting its intended scope.

### Example 1

### Preparation of Dalcetrapib in Liposomes for Oral Application

### Solution A: Mannitol Solution in Distilled Water

7.5 g D-mannitol are weighted into a 200 mL glass flask containing a magnetic stirring bar. 120 mL distilled water are added and the mixture is stirred at room temperature until complete dissolution.

### Solution B: Drug-Lecithin Solution in Ethanol

375 mg dalcetrapib are introduced into a 200 mL glass flask containing a magnetic stirring bar. 7.5 mL ethanol are added and the mixture is stirred until dissolution of the drug. 15.0 g purified egg lecithin (min. 95% phosphatidylcholine content) [Lipoid E100 (Lipoid AG)] are added and stirring is continued until a clear solution is obtained.

Solution A is transferred into solution B while vigorously stirring at about 500 rpm to get a homogenous milky solution. The obtained solution is processed in a high pressure homogenizer (Emulsiflex-C5, Avestin Inc.) under a pressure of about 800 atm.

After a total of 5 cycles, the liposome size is 128 nm (SD 19, n=3) and the final pH is 6.3. The obtained opalescent solution is filtered through a sterile filter of 0.22 μm.

### Example 2

### Preparation of Dalcetrapib in Liposomes for Oral Application

250 mg dalcetrapib are dissolved into a 50 mL flask containing 2.5 mL ethanol. 5.0 g purified egg-lecithin [Lipoid E100 (Lipoid AG)] are added and the mixture is stirred at room temperature at about 20 rpm until complete dissolution.

42.5 mL phosphate buffer pH 7 are added while stirring at about 400 rpm to obtain a homogenous milky colloidal solution.

The obtained solution is processed in a high pressure homogenizer (Emulsiflex-05, Avestin Inc.) under a pressure of ca. 800 atm during 10 min by recycling the solution. The obtained liposomal solution is opalescent, the particle size is 155 nm (SD 14).

### Example 3

### Preparation of Dalcetrapib in Liposomes for Oral Application

Solution A: Mannitol Solution in Distilled. Water

2.5 g D-mannitol are weighted into a 50 mL glass flask containing a magnetic stirring bar. 40 mL distilled water are added and stirred at room temperature until complete dissolution.

### Solution B: Drug-Lecithin Solution in Ethanol

125 mg dalcetrapib are introduced into a 50 mL glass flask containing a magneticstirring bar, 2.5 mL ethanol are added and stirred until dissolution of the drug. 5.0 g purified egg lecithin (min. 95% phosphatidylcholine content) [Lipoid E100 (Lipoid AG)] are added and stirring is continued until a clear solution is obtained.

Solution A is transferred into solution B while vigorously stirring at about 400 rpm to get a homogenous milky solution.

The obtained solution is processed in a high pressure homogenizer (Emulsiflex-05, Avestin Inc.) under a pressure of ca. 800 atm. After 10 min, the particle size is 120 nm (SD 9, n=4) measured by dynamic light scattering (NanoSizer, Malvern) and the final pH is 6.1.

The obtained opalescent solution is filtered through a sterile filter of 0.22 μm.

| | Dalcetrapib | |
|---|---|---|
| | concentration | Particle size |
| Example | (mg/mL) | (PCS, Z-average) |
| 1 | 2.5 | 128 nm (SD 19, n = 4) |
| 2 | 5 | 155 nm (SD 14, n = 4) |
| 3 | 2.5 | 120 nm (SD 9, n = 4) |

### Example 4

### Dalcetrapib at Different Concentrations in 100 mg/mL Liposome Solution

### Solution A: Liposomal Solution Containing Lecithin and Sucrose in Water.

g purified soybean lecithin (min. 95% phosphatidylcholine content) [Lipoid S100 (Lipoid AG)] and 1.5 g D(+)-sucrose are introduced into a 20 mL glass bottle and 7.5 g distilled water are added. The solution is stirred with a magnetic stirring bar at 300 rpm during about 2 hours at room temperature until a homogenous milky multi-lamellar liposome solution is obtained.

The solution is homogenized during 25 minutes with an ultra-sound Sonicator W-375 (Heat Systems Ultrasonics Inc.) having a ½ probe and a cooling water-bath at about 25° C. The obtained opalescent liposome solution is filtered through a 0.45 μm filter and the final particle size is 57 nm.

### Solution B: Dalcetrapib in Ethanol

140 mg of dalcetrapib is dissolved in 1.41 mL of ethanol

Incorporation of Variable Drug Amounts into the Liposomal Solution.

Variable volumes of drug solution B are injected with a pipette into solution A and are shaken during about 10 seconds until total dissolution of the drug.

| Final drug | Solution A | Solution B |
|---|---|---|
| concentration | volume | volume |
| (mg/mL) | (μL) | (μL) |
| 2 | 490 | 10 |
| 3 | 485 | 15 |
| 4 | 480 | 20 |

### Example 5

### 3 mg/mL Dalcetrapib in 100 mg/mL Liposome Lyophilisate

9 mg of dalcetrapib are dissolved in 150 μL of ethanol. 2.85 mL of liposomal solution A from example 4 is added and the mixture is shaken for about 10 seconds. The obtained opalescent solution is frozen by dipping the container with the solution in a mixture of dry ice and ethanol.

The frozen solution is lyophilized in a Christ Beta 2-16 lyophilisator during 22 hours using a predefined lyophilisation cycle.

The particle size changed from 57 nm (SD 6, n=3) before drying to 93 nm (SD 16, n=3) after reconstitution of the lyophilisate with distilled water.

### Example 6

### PK-Study on Cynomolgus Monkeys

The following example provides a pharmacokinetic evaluation of a formulation screening study in the Cynomolgus monkey (n=4). A tablet formulation prepared according to example 1 as disclosed in WO2004082593 was used. This investigation assessed the single dose (10 mg/kg) pharmacokinetics of dalcetrapib in male monkeys following oral dosing by gavage (2.5 mg/mL) with liposome (group 1) or by tablet (group 2) in a regulatory formulation screening study.

### Materials and Methods

The study design was the following:

| | |
|---|---|
| Test animals | Species: Cynomolgus monkey |
| | No. of animals/sex/formulation: male/n = 4 |
| | Surgical intervention: none |
| | Food status: 1 banana offered 30 min before the |
| | compound was applied |
| Test substance | Drug: RO4607381 |
| | Treatment Dose: 10 mg/kg |
| | Administration: oral administration by gavage |
| | or tablet |
| | Frequency: once |
| | Duration: one day |
| Formulation | Description: liposomal solution (G1), Tablet (G2) |
| Pharmacokinetic | Sampling day: Day 1 |
| sampling | Time points: 0.5, 1, 2, 4, 6, 8, 24, 32, 48, and |
| | 56 h postdose |
| | Volume: 0.5 mL |
| | Anticoagulant: EDTA |
| | Sampling method: leg vein |
| | Storage conditions: −80° C. (G1, G2) |
| Bioanalytical | Method: LC-MS/MS |
| Assay | Limit of quantification: 5 ng/mL |

The pharmacokinetic parameters were estimated by non-compartmental analysis, using the pharmacokinetic evaluation program Toxkin™ [1] as follows:

Cmaxand tmax were determined directly from the plasma concentration-time profiles.

AUC(0-24 h) values were calculated by linear trapezoidal rule from time zero to 24 h postdose.

AUC(0-56 h) values were calculated by linear trapezoidal rule from time zero to 56 h postdose.

The concentration at time zero was extrapolated to zero (C(0)=0).

The apparent terminal half-life (t½) was derived from the equation: t½=ln 2/λz.

λz was obtained by log-linear regression of the terminal phase of the plasma concentration-time curve.

For calculation of mean concentration data, values below the limit of quantification and no peak were numerically set to zero. Values of no sample available or invalid data (NOR) were treated as an empty cell in the calculation of means.

Possible small deviations of the reported mean values from those calculated from nonrounded pharmacokinetic parameters are due to the rounding procedure of individual values.

The analytical data are reported with three significant figures. Pharmacokinetic parameters were reported as provided by ToxKin™ (Version 3. 1. 2, Unilog IT Services Ltd., 2004).

| | | | | cₘₐₓ/dose | AUC_{(0-56 h)}/dose |
|---|---|---|---|---|---|
| | Animal | Dose | tₘₐₓ | [ng * kg/ | [ng * h * kg/ |
| Formulation | (monkey) | [mg/kg] | [h] | ml * mg] | ml * mg] |
| Liposomal | Idefix | 10 | 2 | 22.1 | 372 |
| formulation from | James | 10 | 4 | 22.5 | 351 |
| example 1 | Jens | 10 | 2 | 11.3 | 96.7 |
| | Jonas | 10 | 4 | 25.6 | 336 |
| Mean | | | | 20.38 | 288.93 |
| SD | | | | 6.2 | 129.0 |
| Tablet formulation | Idefixs | 10 | 2 | 2.29 | 47.5 |
| according to | James | 10 | 4 | 4.15 | 53.6 |
| example 1 of | Jens | 10 | 0 | — | — |
| WO2004082593 | Jonas | 10 | 2 | 4.67 | 47.8 |
| Mean | | | | 3.7 | 49.63 |
| SD | | | | 1.3 | 3.4 |

The total systemic exposure (AUC(0-56 h), Cmax) was more than 5 times higher in the group administered the liposomal formulation than in the group administered the tablet formulation.

Unless stated to the contrary, all compounds in the examples were prepared and characterized as described. All ranges recited herein encompass all combinations and subcombinations included within that range limit. All patents and publications cited herein are hereby incorporated by reference in their entirety.

1. A composition comprising S-[2-([[1-(2-ethylbutyl)-cyclohexyl]-carbonyl]amino)phenyl]2-methylpropanethioate and liposomes, wherein the S-[2-([[1-(2-ethylbutyl)-cyclohexyl]-carbonyl]amino)phenyl]2-methylpropanethioate is substantially entrapped in a liposome membrane.

2. A composition according to claim 1, wherein at least 95% of S-[2-([[1-(2-ethylbutyl)-cyclohexyl]-carbonyl]amino)phenyl]2-methylpropanethioate in the composition is entrapped in the liposome.

3. A composition according to claim 1, wherein 100% of S-[2-([[1-(2-ethylbutyl)-cyclohexyl]-carbonyl]amino)phenyl]2-methylpropanethioate in the composition is entrapped in the liposome.

4. A composition according to claim 1, wherein the liposomes have sizes of 20 to 1000 nm.

5. A composition according to claim 1, wherein about 80% of the liposomes have sizes of about 25 to about 200 nm.

6. A composition according to claim 1, wherein about 95% of the liposomes have sizes of about 25 to about 200 nm.

7. A composition according to claim 1, wherein the liposomes are vesicles with a membrane comprising a phospholipid bilayer.

8. A composition according to claim 1, wherein the liposomes are spherical vesicles with a membrane comprising a phospholipid bilayer.

9. A composition according to claim 1, wherein the liposomes comprise phospholipids.

10. A composition according claim 9, wherein the phospholipids are selected from the group consisting of egg phosphatidylethanolamine, egg lecithin, dipalmitoyl lecithin, lecithin, egg phosphatidylcholine, dioleoyl phosphatidylcholine, 1-palmitoyl-2-oleoyl-sn-glycerol-3-phosphatidylcholine, dipalmitoylphosphatidylcholine, dimyristoylphosphatidylcholine, and long-chain or intermediate-chain phosphatidylcholine.

11. A composition according to claim 10, wherein the phospholipids are lecithins.

12. A composition according to claim 11, wherein the lecithins are soybean lecithins, egg lecithins or L-β-oleoyl-2-palmitoyl-α-lecithins.

13. A composition according to claim 11, wherein the lecithins are egg lecithins.

14. A composition according to claim 11, wherein the lecithin and at least one stabilizer form the liposome.

15. A composition according to claim 1, wherein the composition comprises at least 40% by weight of water.

16. A composition according to claim 1, wherein 0.01% to 0.5% by weight per volume of S-[2-([[1-(2-ethylbutyl)-cyclohexyl]-carbonyl]amino)phenyl]2-methylpropanethioate is present.

17. A composition according to claim 1, wherein 0.1% to 0.3% weight per volume of S-[2-([[1-(2-ethylbutyl)-cyclohexyl]-carbonyl]amino)phenyl]2-methylpropanethioate is present.

18. A composition according to claim 1, wherein 0.25% by weight per volume, of S-[2-([[1-(2-ethylbutyl)-cyclohexyl]-carbonyl]amino)phenyl]2-methylpropanethioate is present.

19. A composition according to claim 1, wherein the concentration of the liposome component in the solution is about 1% to about 25% (weight/volume).

20. A composition according to claim 1, wherein the composition is in the form of a solution.

21. A kit comprising:
a solution of S-[2-([[1-(2-ethylbutyl)-cyclohexyl]-carbonyl]amino)phenyl]2-methylpropanethioate in alcohol; and
a solution of liposome.

22. The kit of claim 21, wherein the solution of liposomes is an aqueous solution.

23. The kit of claim 21 comprising:
a vial with a solution of S-[2-([[1-(2-ethylbutyl)-cyclohexyl]-carbonyl]amino)phenyl]2-methylpropanethioate in a pharmaceutically acceptable solvent; and
a vial with a solution of liposome.

24. The kit of claim 21 comprising:
a vial with a solution of S-[2-([[1-(2-ethylbutyl)-cyclohexyl]-carbonyl]amino)phenyl]2-methylpropanethioate in a pharmaceutically acceptable solvent; and
a vial with a solution of liposome in water.

25. The kit of claim 21, wherein lecithin and at least one stabilizer form the liposome,

26. The kit of claim 21, wherein the alcohol is ethanol.

27. The kit of claim 25, wherein the stabilizer is a carbohydrate.

28. The kit of claim 25, wherein the stabilizer is a monosaccharide, disaccharide, or a sugar-like polyol.

29. The kit of claim 25, wherein the stabilizer is glucose, fructose, sucrose, sorbitol, mannitol or xylitol.

30. The kit of claim 21, wherein the stabilizers include 1% to 25% by weight per volume of carbohydrates, 0.5% to 3% by weight per volume charged phospholipids, or cholesterol.

31. A method of preparing a liposome composition comprising:
a) preparing an oil-soluble composition comprising S-[2-([[1-(2-ethylbutyl)-cyclohexyl]-carbonyl]amino)phenyl]2-methylpropanethioate and lecithin in a pharmaceutically acceptable solvent;
b) preparing a water soluble composition comprising a stabilizer and water;
c) combining the water soluble composition with the oil-soluble composition obtained according to step b) and a) respectively;
d) stirring; and
e) homogenizing at high pressure between 20 to 150 MPa.
